SEQUENCE LISTING (9 sequences)

SEQ ID NO: 1 - DNA, 22 bp, Artificial Sequence
  Primer
gtaactcagcagcatctcaggg

SEQ ID NO: 2 - DNA, 24 bp, Artificial Sequence
  Primer
aggtgattttggtctagctacaga

SEQ ID NO: 3 - DNA, 23 bp, Artificial Sequence
  Primer
ggtgattttggtctagctacaga

SEQ ID NO: 4 - DNA, 24 bp, Artificial Sequence
  Primer
aggtgattttggtctagctaccga

SEQ ID NO: 5 - DNA, 23 bp, Artificial Sequence
  Primer
ggtgattttggtctagctaccga

SEQ ID NO: 6 - DNA, 24 bp, Artificial Sequence
  Primer
aggtgattttggtctagctacagt

SEQ ID NO: 7 - DNA, 23 bp, Artificial Sequence
  Primer
ggtgattttggtctagctacagt

SEQ ID NO: 8 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 9 - protein, 766 aa, Homo sapiens [sequence omitted]

(19) United States
(12) Patent Application Publication
(10) Pub. No.: US 2013/0130264 A1
(43) Pub. Date: May 23, 2013

(54) Methods of Detecting BRAF Mutations in Cancer

(71) Applicants: Enrico Tiacci, Perugia (IT); Brunangelo Falini, Perugia (IT)

(72) Inventors: Enrico Tiacci, Perugia (IT); Brunangelo Falini, Perugia (IT)

(21) Appl. No.: 13/659,895

(22) Filed: Oct. 24, 2012

Related U.S. Application Data
(60) Provisional application No. 61/550,504, filed on Oct. 24, 2011.

(51) Int. Cl. C12Q 1/68

(52) U.S. Cl. CPC C12Q 1/6886
USPC 435/6.12

(57) ABSTRACT

The present disclosure relates to detecting BRAF mutations and methods of utilizing BRAF mutations to diagnose cancer.

### RELATED APPLICATIONS

This application claims benefit of priority from U.S. Provisional Patent Application 61/550,504, filed Oct. 24, 2011, which is hereby incorporated in its entirety as if fully set forth.

### FIELD OF THE DISCLOSURE

This disclosure relates to detecting BRAF mutations as well as methods of, and kits for, utilizing BRAF mutations to diagnose cancer.

### BACKGROUND OF THE DISCLOSURE

The uncontrolled growth of abnormal cells in the body can form either a benign or malignant tumor. When these abnormally proliferating cells are malignant, then they are diagnosed as cancer. Malignancy in cancers is characterized by anaplasia (reversion of differentiated cells to a less differentiated, or more stem cell-like phenotype), invasiveness, and metastasis (spread of the cancerous cells from one tissue or organ, or one part of the body, to another non-adjacent tissue, organ or part of the body). Although cancers share many common features, treatments that are tailored to the root causes of the cancer are often most successful.

Hairy cell leukemia (HCL) is a chronic hematological malignancy characterized by an accumulation of abnormal B lymphocytes. HCL was originally described as histiocytic leukemia, malignant reticulosis, or lymphoid myelofibrosis. The disease was formally named leukemic reticuloendotheliosis and its common name is derived from the “hairy” appearance of the malignant B cells under a microscope.

It is essential to distinguish HCL from other lymphoid malignancies that can masquerade as this disease (e.g., hairy cell variant; splenic marginal zone lymphoma, splenic diffuse red pulp small B-cell lymphoma, splenic leukemia/lymphoma unclassifiable, chronic lymphocytic leukemia, prolymphocytic leukemia, other low grade lymphomas, and systemic mastocytosis). HCL diagnosis is currently based on a combination of methodologies including, physical examination, complete blood count (cbc), peripheral blood smears and bone marrow biopsy in conjunction with light microscopy, flow cytometry and immunohistochemistry. However, none of these tests can accurately diagnose HCL in all instances.

Accordingly, there are benefits in identifying genes and proteins which may be dysregulated during the development and progression of all cancers, and, in particular, hairy cell leukemia, and to utilize these genes and proteins as biomarkers for disease development and progression and therapeutic treatment efficacy.

### SUMMARY OF THE DISCLOSURE

Hairy cell leukemia (HCL) is a distinct clinico-pathological entity. HCL responds well to treatment with purine analogs, however, HCL is difficult to differentiate from other HCL-like disorders (e.g., splenic marginal zone lymphoma and HCL-variant). The BRAF V600E mutation was identified as the disease-defining genetic event in HCL. In a first aspect, this disclosure provides a novel, simple, and inexpensive test for a genetics-based method diagnosis of HCL using a nucleic acid containing sample, such as samples of whole-blood as non-limiting examples. The method detects the BRAF-V600E through a sensitive allele-specific polymerase-chain reaction (PCR) qualitative assay. In some embodiments, the assay may be followed by agarose-gel electrophoresis. Using these methods, BRAF-V600E was detected in 113 of 113 leukemic HCL samples investigated. Some of these samples contained as few as 0.2% leukemic cells, demonstrating that this method is extremely sensitive and does not require a significant number of cancer cells (e.g. leukemic cells) to be effective. Thus, this method is effective for detection of early-stage cancer or those cancers entering remission.

BRAF-V600E was detected at different time points during the disease course, even at post-therapy time points, demonstrating the pivotal role of this mutation in HCL pathogenesis and maintenance of the leukemic clone. Conversely, 111 non-HCL chronic B-cell neoplasms, including 75 HCL-like disorders, were invariably negative for BRAF-V600E. The molecular assay is a powerful tool for improving the diagnostic accuracy in cancer, and, in particular, in HCL.

In another aspect, the present disclosure provides, in part, a method of diagnosing hairy cell leukemia in a subject in need thereof. In one embodiment, the method includes: obtaining a biological sample from the subject and assessing the presence or absence of a BRAF mutation in the sample, wherein the presence of the BRAF mutation indicates that the subject is suffering from hairy cell leukemia.

In some cases, the method can further include comparing the presence, absence, or amount of the BRAF mutation in the biological sample with the presence, absence, or amount of the BRAF mutation determined in a biological sample from a subject not suffering from cancer or symptoms thereof. In other embodiments, the method can be used to distinguish hairy cancer cells from non-cancer cells.

In a further aspect, the disclosure provides a method, comprising (a) extracting at least one DNA molecule from a biological sample from a subject; (b) amplifying said DNA molecule by polymerase chain reaction (PCR) using a combination of a reverse primer comprising the sequence 5′-GTAACTCAGCAGCATCTCAGGG-3′ (SEQ ID NO: 1) and a forward primer comprising one of the following sequences:

(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,

5′-GGTGATTTTGGTCTAGCTACCGA-3′ (SEQ ID NO: 5), wherein the amplification product comprises a mutation in BRAF; optionally (c) comparing the amplified product of step (b) with a control or wild type amplification product of BRAF; (d) determining the presence or absence of the mutation in BRAF in the subject; and (e) diagnosing or identifying the subject as having cancer if the mutation is present. In some embodiments, the method is performed in the presence of wild-type BRAF nucleic acid molecules (BRAF nucleic acid molecules that are non-mutant at the position corresponding to amino acid residue 600 of SEQ ID NO:9 or the codon corresponding to positions 1859-1861 of SEQ ID NO:8).

In another embodiment of this method, the control or wild type amplification product of BRAF is amplified using a combination of a forward primer comprising either the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7), and the reverse primer comprising the sequence of 5′-GTAACTCAGCAGCATCTCAGGG-3′ (SEQ ID NO: 1). The control or wild type amplification product of BRAF includes a thymine (T) nucleotide at position 1860 of SEQ ID NO: 8. Alternatively, or in addition, the control or wild type amplification product of BRAF encodes for a Valine (Val or V) residue at amino acid residue 600 of SEQ ID NO: 9.

The mutation in the human BRAF gene is a substitution of an adenine (A) for a thymine (T) nucleotide at position 1860 of SEQ ID NO: 8. The mutation in the human BRAF gene encodes for a mutation in the resultant amino acid sequence, wherein a glutamic acid (Glu or E) is substituted for a Valine (Val or V) residue at amino acid residue 600 of SEQ ID NO: 9, i.e. the V600E substitution. This BRAF mutation is also referred to herein as the “BRAF V600E” mutation.

In some embodiments of the disclosed methods, the amplified or detected DNA molecule is genomic DNA. In other embodiments, the amplified or detected molecule is a cDNA. An exemplary cDNA is one produced from a BRAF encoding RNA or mRNA.

In some embodiments, the biological sample is a tissue sample or a bodily fluid. Non-limiting examples of a tissue sample include, but are not limited to, bone marrow and spleen. Non-limiting examples of a bodily fluid include, but are not limited to, peripheral blood.

In some cases, the subject may be diagnosed with cancer, while in other cases the subject may not be diagnosed with cancer. In some embodiments, the subject has cancer, but has not been diagnosed because the methods of the disclosure are used in the diagnosis or prognosis process. Subjects may be of any age, including, but not limited to infants, toddlers, children, minors, adults, seniors, and elderly individuals.

A subject of the disclosure may have any type or severity of cancer. In some embodiments, the cancer is primary or metastatic cancer. Moreover, the cancer is a solid or liquid cancer. Non-limiting examples of cancer include, but are not limited to, adrenal cortical cancer, anal cancer, bile duct cancer, bladder cancer, bone cancer, brain or a nervous system cancer, breast cancer, cervical cancer, colon cancer, rectral cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), hairy cell leukemia (HCL), non-HCL lymphoid malignancy (hairy cell variant, splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, or splenic lymphoma/leukemia unclassifiable (SLLU)), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, and Wilms Tumor.

In some cases, the cancer is hairy cell leukemia (HCL). In other cases, the cancer is a non-HCL lymphoid malignancy. Non-limiting examples of non-HCL lymphoid malignancy include, but are not limited to, hairy cell variant (HCL-v), splenic marginal zone lymphoma (SMZL), splenic diffuse red pulp small B-cell lymphoma (SDRPSBCL), splenic leukemia/lymphoma unclassifiable (SLLU), chronic lymphocytic leukemia (CLL), prolymphocytic leukemia, low grade lymphoma, systemic mastocytosis, and splenic lymphoma/leukemia unclassifiable (SLLU).

The disclosure further describes a kit for detecting the presence of a BRAF mutation in a biological sample, comprising (a) a forward primer comprising one of the following sequences:

(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,

5′-GGTGATTTTGGTCTAGCTACCGA-3′ (SEQ ID NO: 5); (b) a reverse primer comprising the sequence 5′-GTAACTCAGCAGCATCTCAGGG-3′ (SEQ ID NO: 1) and (c) instructions for detecting the presence of a BRAF mutation in the biological sample in a manner disclosed herein. In some embodiments, the kit further comprises a forward primer comprising either the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7). In alternative embodiments, a kit may comprise the above combinations of forward and reverse primers in one or more containers with labels directing their use in a method disclosed herein.

In additional embodiments, the disclosure provides a kit for detecting the presence of a BRAF mutation in a biological sample, comprising (a) a forward primer comprising one of the following sequences:

(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,

5′-GGTGATTTTGGTCTAGCTACCGA-3′ (SEQ ID NO: 5); (b) a reverse primer comprising the sequence of 5′-GTAACTCAGCAGCATCTCAGGG-3′ (SEQ ID NO: 1) and (c) instructions for carrying out or performing a method described herein. In other embodiments, a kit further comprises a forward primer comprising the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7). In alternative embodiments, a kit may comprise the above combinations of forward and reverse primers in one or more containers with labels directing their use in a method disclosed herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. In addition, the materials, methods, and examples are illustrative only and are not intended to be limiting.

Other features and advantages of the disclosure will be apparent from the following detailed description and claims.

### BRIEF DESCRIPTION OF THE DRAWINGS

FIG. 1A is a flow cytometry dot plot of a whole-blood sample subjected to red blood cell lysis from a representative HCL patient. HCL cells (CD19+/CD25+ red (boxed) events in FIG. 1B) represent 2% of all nucleated cells (CD45+ black (boxed) events). HCL-v cells (CD19+/CD25− and CD19+/CD103+ red (boxed) events in FIG. 1C and FIG. 1D, respectively) represent 92% of all cells.

FIG. 1B is a flow cytometry dot plot of a whole-blood sample subjected to red blood cell lysis from a representative HCL patient. HCL cells (CD19+/CD25+ red (boxed) events) represent 2% of all nucleated cells (CD45+ black (boxed) events in FIG. 1A). HCL-v cells (CD19+/CD25− and CD19+/CD103+ red (boxed) events in FIG. 1C and FIG. 1D, respectively) represent 92% of all cells.

FIG. 1C is a flow cytometry dot plot of purified peripheral blood leukemic cells from a representative patient with HCL-v. HCL cells (CD19+/CD25+ red (boxed) events in FIG. 1B) represent 2% of all nucleated cells (CD45+ black (boxed) events in FIG. 1A). HCL-v cells (CD19+/CD25− and CD19+/CD103+ red (boxed) events in FIG. 1C and FIG. 1D, respectively) represent 92% of all cells.

FIG. 1D is a flow cytometry dot plot of purified peripheral blood leukemic cells from a representative patient with HCL-v. HCL cells (CD19+/CD25+ red (boxed) events in FIG. 1B) represent 2% of all nucleated cells (CD45+ black (boxed) events in FIG. 1A). HCL-v cells (CD19+/CD25− and CD19+/CD103+ red (boxed) events in FIG. 1C and FIG. 1D, respectively) represent 92% of all cells.

FIG. 1E is a photograph of a conventional agarose-gel electrophoresis of samples from 13 HCL patients (12 pre-treatment, 1 with Minimal Residual Disease—MRD—post-treatment), after allele-specific (AS)-PCR for the mutant allele (top panel) and for the wild-type allele (bottom panel). Serial dilutions of mutated and wild-type alleles (from 3.1% to 0%) are also included to show the analytical sensitivity of the mutant-AS-PCR (_{—}0.1% mutated alleles). All HCL samples gave rise to a mutant BRAF-V600E band. In contrast, none of the HCL-like samples gave rise to a mutant BRAF-V600E band. To facilitate the visualization of the results, the gel lane of HCL case 13 was repositioned.

FIG. 1F is a photograph of a conventional agarose-gel electrophoresis of samples from 16 HCL-like patients (6 SLLU, 10 SMZL), after AS-PCR for the mutant allele (top panel) and for the wild-type allele (bottom panel). None of the HCL-like samples gave rise to a mutant BRAF-V600E band. SMZL case 30, which did not give rise to the wild-type band, was not evaluable in this particular experiment (shown on purpose), but upon repetition turned out to be evaluable (i.e., provided a strong wild-type band). SMZL case 30 is also negative for BRAFV600E (i.e., mutant band not visible). To facilitate the visualization of the results, the gel lane of the 50-bp DNA ladder was repositioned.

### DETAILED DESCRIPTION OF MODES OF PRACTICING THE DISCLOSURE

Hairy cell leukemia (HCL) is a slow growing cancer of mature B-cell origin. The disease involves the bone marrow, liver, spleen and sometimes lymph nodes. The symptoms seen in patients with HCL are varied and reflect both direct involvement in organs, secondary effects on the immune system, and release of cytokines or proteins from the malignant cell itself.

Hairy cell leukemia (HCL) is considered a distinct entity within the field of cancer or leukemia, which is usually characterized by splenomegaly (usually without lymphadenopathy), pancytopenia, and infiltration of bone marrow, spleen and liver by leukemic B cells with “hairy” appearance. In contrast to other chronic B-cell leukemias, HCL cells circulate at low percentages in the blood (Foucar K., et al. Hairy cell leukaemia. In: Swerdlow S, et al., eds. WHO Classification of Tumours of Haematopoietic and Lymphoid Tissues (4th edition). Lyon: International Agency for Research on Cancer (IARC), Lyon, France; 2008: 188-190), and exhibit distinct functional features and gene expression profile (Basso K, et al. J Exp Med. 2004; 199:59-68; Tiacci E, et al. Nat Rev Cancer. 2006; 6:437-448).

The most common complications seen involve the blood, the bone marrow and the spleen. The effects on the blood and the bone marrow are as follows: The majority of patients will have some degree of reduced blood count with about 40% of patients having depression of all blood cell lines or pancytopenia. In looking at the individual cell lines in large retrospective series anemia as defined by a hemoglobin less than 12 grams/dl is seen in up to 80% of patients with severe anemia with hemoglobin less than 8.5 grams/dl in about one third of patients. This significant anemia may lead to fatigue and reduced exercise tolerance and is often the first symptoms of this disease. The cause of the anemia may be multifactorial including iron deficiency from blood loss and occasionally autoimmune hemolytic anemia. However, the common reason for the anemia is removal of red blood cells in the spleen and marrow infiltration with hairy cells leading to reduced red cell production. Thrombocytopenia is a frequent complication of this disease with platelets less than 100,000/μl in up to 80% of patients. Severe thrombocytopenia of less than 50,000/μl occurs in about one third of patients with about 10% having counts under 20,000/μl. Significant bleeding is usually only seen in severely depressed platelet counts.

The spleen appears to play a significant role, since platelets return to normal after splenectomy in 70% of patients and is especially important in patients with large spleens. However, post splenectomy patients do develop thrombocytopenia due to hairy cell involvement in the marrow and occasionally immune thrombocytopenia is seen.

Leucopenia and neutropenia is one common reason to suspect HCL and leads to one the most severe complications that of significant infections. Life threatening neutropenia with neutrophils of under 500/μl occurs in almost 40% of patients. This depressed white count will often be improved by the use of granulocyte growth factors. An additional diagnostic finding is the presence of marked monocytopenia with resultant susceptibility to unusual organisms.

Hepatomegaly is much less frequent in hairy cell patients with enlargement noted about one third of the time and marked hepatomegaly or greater than 10 cm below the costal margin only 2% of the time. Pain from this hepatomegaly is not common but can occur. The liver is almost always infiltrated with hairy cells without significantly altering hepatic function or elevating liver enzymes. The development of marked hyperbilirubinemia and elevated liver enzyme elevations does occur, but its rarity should make one consider an infectious etiology. In addition, one can see portal hypertension due to involvement with subsequent ascites.

Splenomegaly is one the classic findings found at presentation in patients with hairy cell leukemia. On physical examination up to 90% of patients will have an enlarged spleen and marked splenomegaly of greater than 10 cm below the costal margin seen in 20% of patients. The enlarged spleen may cause early satiety with subsequent weight loss and can be associated with painful splenic infarction or splenic rupture.

One of the most recognized and important, clinical problem in patients with HCL is the development of severe life threatening and unusual infections. These may involve the common sites of lung and urinary tract as well as less common involvement of the liver and central nervous system. Patients may develop a wide range of infections including those usually seen in the neutropenic host such as staphylococcus aureus. Pseudomonas aeruginosa Herpes zoster with painful skin lesions is usually only seen after patients have been treated with chemotherapy. Patients with fever of unknown origin should always be treated as if they have a significant infection and a careful search for bacterial, fungal, or viral infection be initiated.

Several other unusual complications can be seen. Neurologic complications including symptoms and signs of meningitis and nerve compression has been reported but one should always look for infection as a cause. Lymphadenopathy is infrequent and when it is present usually involves the chest or abdominal nodes. These can be bulky and cause symptoms of compression. Destructive bone lesions with severe pain can be seen usually in long bones or vertebrae. Finally, involvement of the lining of the lung cavity or pleura or that of the abdominal cavity or peritoneal surface can lead to accumulation of fluid in these areas with symptoms of abdominal pain and or shortness of breath.

Accurate diagnosis of HCL is important since very effective therapy used for treatment of HCL is much less effective in other types of chronic B cell lymphoproliferative disorders. Diagnosis is currently established based on a combination of morphologic and immunophenotypic findings. Blood smear, bone marrow aspirate smears, bone marrow touch preparations and bone marrow biopsy are most often used for diagnosis of HCL. If available; spleen, liver biopsy or rarely other tissue involved by HCL may be used for diagnosis of HCL as well. Furthermore, HCL diagnosis relies on morphological and immunophenotypic criteria (Greyer M R. Blood. 2010; 115:21-28) that usually allow its distinction from HCL-like disorders of the 2008-World Health Organization (WHO) classification, i.e. splenic marginal zone lymphoma (SMZL) and splenic lymphoma/leukemia unclassifiable (SLLU, which includes HCL-variant—HCL-v) (Piris M, et al. Splenic B-cell lymphoma/leukaemia, unclassificable. In: Swerdlow S, et al., eds. WHO classification of tumours of haematopoietic and lymphoid tissues (4th edition). Lyon: International Agency for Research on Cancer (IARC); 2008: 191-193).

The most problematic potential cases of HCL can be diagnosed using Annexin-A1 immunostaining (Falini B, et al. Lancet. 2004; 363:1869-1870; Dong H Y, et al. Am J Clin Pathol. 2009; 131:586-595; Sadik W, et al. Br J Haematol. 2010; 151:207), which was previously reported to be highly sensitive and specific for HCL among B-cell lymphomas (Falini B, et al. Lancet. 2004; 363:1869-1870). However, because Annexin-A1 is also expressed by myeloid and T cells (Falini B, et al. Lancet. 2004; 363:1869-1870), this immunohistochemical staining may be difficult to interpret in bone marrow biopsies with low percentages of HCL cells. Moreover, immunocytochemistry for Annexin-A1 is not readily applicable to routine hematological samples, such as peripheral blood or diluted bone marrow aspirate (due to HCL-induced marrow fibrosis and consequent dry tap), that are also usually poor in HCL cells and rich in neuthrophils and T cells.

In spite of the remarkable progress in the diagnosis and treatment of HCL over the past 50 years, its underlying genetic alterations remain obscure (Tiacci et al., Nat Rev Cancer 2006; 6(6):437-48). Major obstacles to molecular characterization of HCL have been the scarcity of tumor cells available for analysis (due to frequent pancytopenia), the very low proliferative index of leukemic cells, the inability to grow them in immunodeficient mice and the absence of human cell lines of authentic HCL origin.

No recurrent chromosomal translocations have been identified in HCL (Foucar, et al., WHO Classification of Tumours of Haematopoietic and Lymphoid Tissues. 4th ed. Lyon: International Agency for Research on Cancer (IARC); 2008: 188-90). Gene expression profiling studies revealed a unique molecular signature that in part justifies the distinctive features of HCL cells, like their morphological appearance, adhesion properties, selective homing to extranodal sites and marrow fibrosis (Basso, et al. J Exp Med 2004; 199(1):59-68). However, these studies did not pinpoint any recurrent genetic alteration. Similarly, high density genome-wide SNP genotyping showed a remarkably balanced genomic profile in HCL (Forconi, et al. Br J Haematol 2008; 141(5):622-30).

One solution for accurate diagnosis of HCL is a sensitive and specific method for a genetics-based diagnosis of HCL. BRAF-V600E was recently identified as the HCL-defining genetic lesion (present in all HCL cases, absent in other B-cell neoplasms) (Tiacci E, et al. N Engl J Med. 2011; 364:2305-2315) by Sanger sequencing of BRAF exon-15. However, this technique required ≧30% leukemic cells for reliably detecting a clonal heterozygous mutation. Thus, the rare HCL cells typically present in the blood of most patients had to be purified through cell sorting (Tiacci E, et al. N Engl J Med. 2011; 364:2305-2315), a laborious procedure not amenable to a routine diagnostic setting.

Thus, the disclosure provides a sensitive, easy and inexpensive test for the routine clinical diagnosis of HCL in, for instance, blood samples. In some embodiments, the test is based on BRAF-V600E detection by allele-specific PCR (AS-PCR) followed, for example, by conventional agarose-gel electrophoresis. The examples provided herein demonstrate the diagnostic accuracy of this test in a large cohort of HCL and HCL-like disorders. In other embodiments, the test may be based upon quantitative or real-time PCR as known and practiced by the skilled person.

The disclosed methods identified the BRAF V600E mutation as a genetic alteration recurrently associated with HCL. The BRAF V600E mutation qualifies as a disease-defining genetic event in HCL because of: i) its presence in 100% of cases encompassing the whole spectrum of HCL patients, including those presenting with leukocytosis or without splenomegaly and those analyzed after therapy; ii) its presence in the entire tumor cell clone in virtually all patients; and iii) its restriction to HCL among peripheral B-cell lymphomas/leukemias. This demonstrates the BRAF V600E mutation in HCL pathogenesis. Notably, among B-cell neoplasms (in which non-kinase genes are usually involved by a variety of genetic alterations, i.e. translocations, deletions, or point mutations), HCL is the only one whose disease-defining genetic lesion is represented by an activating point mutation of a kinase-encoding gene. Surprisingly, the frequency of BRAF V600E in HCL far outnumbers that previously reported for other BRAF-mutated human neoplasms, including melanomas (˜50%) (Davies, et al. Nature 2002; 417(6892):949-54; urtin, et al. N Engl J Med 2005; 353(20):2135-47), papillary thyroid carcinomas (˜40%) (Puxeddu, et al. J Clin Endocrinol Metab 2004; 89(5):2414-20), Langherans cell histiocytosis (57%) (Badalian-Very, et al. Blood; 116(11):1919-23) and a variety of solid tumors (at much lower frequency) (Davies, et al. Nature 2002; 417(6892):949-54; Brose, et al. Cancer Res 2002; 62(23):6997-7000; Tie, et al. Int J Cancer 2011 May 1; 128(9):2075-84).

A member of the serine/threonine kinase RAF family, the BRAF protein is part of the RAS-RAF-MAPK signaling pathway that plays a major role in regulating cell survival, proliferation and differentiation (Keshet and Seger. Methods Mol Biol; 661:3-38). BRAF mutations constitutively activate the MEK-ERK pathway, leading to enhanced cell proliferation, survival and ultimately, neoplastic transformation (Wellbrock and Hurlstone. Biochem Pharmacol; 80(5):561-7; Li et al. Oncol Rep 2009; 22(4):671-81; Niault and Baccarini. Carcinogenesis; 31(7):1165-74). All BRAF mutated HCL cases carried the V600E phospho-mimetic substitution which occurs within the BRAF activation segment and markedly enhances its kinase activity in a constitutive manner (Wan, et al. Cell 2004; 116(6):855-67).

The BRAF V600E mutation accounts for some HCL immunophenotypic features, e.g. the low/moderate cyclin D1 expression (which is independent of CCND1 rearrangements or amplifications) (Bosch, et al. Br J Haematol 1995; 91(4):1025-30; Miranda, et al. Mod Pathol 2000;13(12):1308-14) and absence of p27 (Chilosi, et al. Br J Haematol 2000; 111(1):263-71). In melanoma cells, V600E BRAF leads to MEK/ERK pathway activation with concomitant transcriptional constitutive expression of cyclin D1 and p27 down-regulation in an adhesion-independent manner (Roovers, et al. Mol Biol Cell 1999; 10(10):3197-204; Bhatt et al. Oncogene 2005; 24(21):3459-71; Bhatt et al. Oncogene 2007; 26(7):1056-66). Moreover, MEK-ERK-induced activation of an AP1-transcription factor complex containing JUND (Nicolaou, et al. Blood 2003; 101(10):4033-41) has been implicated in the expression of the HCL marker CD11c.

In studies that led to the identification of BRAF V600E, the mutation was present in all 47 HCL cases analyzed. Among a total of 240 peripheral B-cell lymphomas studied, BRAF V600E was restricted to HCL. Subsequent studies (see below the “Examples” section) extended this result to a larger number of patients.

The disclosure demonstrates that BRAF mutations, such as BRAF V600E, can be readily utilized as a diagnostic biomarker of cancer. In some embodiments, BRAF mutations, such as BRAF V600E, can be readily utilized as a diagnostic biomarker to distinguish HCL from other B-cell lymphomas exhibiting similar clinical and morphological features, such as HCL-variant and splenic marginal zone lymphoma, none of which were positive for BRAF-mutations. This distinction is critically relevant clinically since HCL but not HCL-like disorders respond optimally to interferon or purine analogs (Greyer M R. Blood; 115(1):21-8). Absence of BRAF mutations in HCL-variant further supports the view that this entity is different from HCL and justifies its inclusion in the category of splenic B-cell lymphoma/leukemia, unclassifiable in the 2008 WHO classification (Phis et al. WHO classification of tumours of haematopoietic and lymphoid tissues. 4th edition ed. Lyon: International Agency for Research on Cancer (IARC); 2008).

The present disclosure provides, in part, a method of diagnosing hairy cell leukemia in a subject in need thereof including: obtaining a biological sample from the subject and assessing the presence or absence of a BRAF mutation in the sample, wherein the presence of the BRAF mutation indicates that the subject is suffering from hairy cell leukemia.

In some embodiments, the method can further include comparing the presence, absence, or amount of the BRAF mutation in the biological sample with the presence, absence, or amount of the BRAF mutation determined in a biological sample from a subject not suffering from hairy cell leukemia or symptoms thereof. The method can be used to distinguish hairy cell leukemia cells from other forms of malignant lymphoma.

In many cases, the BRAF mutation is a BRAF V600E mutation, in which a glutamic acid (Glu or E) is substituted for a Valine (Val or V) residue at position or amino acid residue 600 of SEQ ID NO: 9. Alternatively, or in addition, the BRAF mutation is a substitution of an adenine (A) for a thymine (T) nucleotide at position 1860 of SEQ ID NO: 8.

Homo sapiens v-raf murine sarcoma viral oncogene homolog B1, BRAF, is encoded by the following mRNA sequence (NM_{—}004333, SEQ ID NO: 8) (wherein coding sequence is bolded and the coding sequence for amino acid residue 600 is underlined and enlarged):

```
      1 cgcctccctt ccccctcccc gcccgacagc ggccgctcgg gccccggctc tcggttataa
    61 gatggcggcg ctgagcggtg gcggtggtgg cggcgcggag ccgggccagg ctctgttcaa
  121 cggggacatg gagcccgagg ccggcgccgg cgccggcgcc gcggcctctt cggctgcgga
  181 ccctgccatt ccggaggagg tgtggaatat caaacaaatg attaagttga cacaggaaca
  241 tatagaggcc ctattggaca aatttggtgg ggagcataat ccaccatcaa tatatctgga
  301 ggcctatgaa gaatacacca gcaagctaga tgcactccaa caaagagaac aacagttatt
  361 ggaatctctg gggaacggaa ctgatttttc tgtttctagc tctgcatcaa tggataccgt
  421 tacatcttct tcctcttcta gcctttcagt gctaccttca tctctttcag tttttcaaaa
  481 tcccacagat gtggcacgga gcaaccccaa gtcaccacaa aaacctatcg ttagagtctt
  541 cctgcccaac aaacagagga cagtggtacc tgcaaggtgt ggagttacag tccgagacag
  601 tctaaagaaa gcactgatga tgagaggtct aatcccagag tgctgtgctg tttacagaat
  661 tcaggatgga gagaagaaac caattggttg ggacactgat atttcctggc ttactggaga
  721 agaattgcat gtggaagtgt tggagaatgt tccacttaca acacacaact ttgtacgaaa
  781 aacgtttttc accttagcat tttgtgactt ttgtcgaaag ctgcttttcc agggtttccg
  841 ctgtcaaaca tgtggttata aatttcacca gcgttgtagt acagaagttc cactgatgtg
  901 tgttaattat gaccaacttg atttgctgtt tgtctccaag ttctttgaac accacccaat
  961 accacaggaa gaggcgtcct tagcagagac tgccctaaca tctggatcat ccccttccgc
1021 acccgcctcg gactctattg ggccccaaat tctcaccagt ccgtctcctt caaaatccat
1081 tccaattcca cagcccttcc gaccagcaga tgaagatcat cgaaatcaat ttgggcaacg
1141 agaccgatcc tcatcagctc ccaatgtgca tataaacaca atagaacctg tcaatattga
1201 tgacttgatt agagaccaag gatttcgtgg tgatggagga tcaaccacag gtttgtctgc
1261 taccccccct gcctcattac ctggctcact aactaacgtg aaagccttac agaaatctcc
1321 aggacctcag cgagaaagga agtcatcttc atcctcagaa gacaggaatc gaatgaaaac
1381 acttggtaga cgggactcga gtgatgattg ggagattcct gatgggcaga ttacagtggg
1441 acaaagaatt ggatctggat catttggaac agtctacaag ggaaagtggc atggtgatgt
1501 ggcagtgaaa atgttgaatg tgacagcacc tacacctcag cagttacaag ccttcaaaaa
1561 tgaagtagga gtactcagga aaacacgaca tgtgaatatc ctactcttca tgggctattc
1621 cacaaagcca caactggcta ttgttaccca gtggtgtgag ggctccagct tgtatcacca
1681 tctccatatc attgagacca aatttgagat gatcaaactt atagatattg cacgacagac
1741 tgcacagggc atggattact tacacgccaa gtcaatcatc cacagagacc tcaagagtaa
1801 taatatattt cttcatgaag acctcacagt aaaaataggt gattttggtc tagctacagt
1861 gaaatctcga tggagtgggt cccatcagtt tgaacagttg tctggatcca ttttgtggat
1921 ggcaccagaa gtcatcagaa tgcaagataa aaatccatac agctttcagt cagatgtata
1981 tgcatttgga attgttctgt atgaattgat gactggacag ttaccttatt caaacatcaa
2041 caacagggac cagataattt ttatggtggg acgaggatac ctgtctccag atctcagtaa
2101 ggtacggagt aactgtccaa aagccatgaa gagattaatg gcagagtgcc tcaaaaagaa
2161 aagagatgag agaccactct ttccccaaat tctcgcctct attgagctgc tggcccgctc
2221 attgccaaaa attcaccgca gtgcatcaga accctccttg aatcgggctg gtttccaaac
2281 agaggatttt agtctatatg cttgtgcttc tccaaaaaca cccatccagg cagggggata
2341 tggtgcgttt cctgtccact gaaacaaatg agtgagagag ttcaggagag tagcaacaaa
2401 aggaaaataa atgaacatat gtttgcttat atgttaaatt gaataaaata ctctcttttt
2461 ttttaaggtg aaccaaagaa cacttgtgtg gttaaagact agatataatt tttccccaaa
2521 ctaaaattta tacttaacat tggattttta acatccaagg gttaaaatac atagacattg
2581 ctaaaaattg gcagagcctc ttctagaggc tttactttct gttccgggtt tgtatcattc
2641 acttggttat tttaagtagt aaacttcagt ttctcatgca acttttgttg ccagctatca
2701 catgtccact agggactcca gaagaagacc ctacctatgc ctgtgtttgc aggtgagaag
2761 ttggcagtcg gttagcctgg gttagataag gcaaactgaa cagatctaat ttaggaagtc
2821 agtagaattt aataattcta ttattattct taataatttt tctataacta tttcttttta
2881 taacaatttg gaaaatgtgg atgtctttta tttccttgaa gcaataaact aagtttcttt
2941 ttataaaaa
```

Homo sapiens v-raf murine sarcoma viral oncogene homolog B1, BRAF, is encoded by the following amino acid sequence (NP_{—}004324, SEQ ID NO: 9) (wherein amino acid residue 600 is bolded and underlined and enlarged):

```
    1 maalsggggg gaepgqalfn gdmepeagag agaaassaad paipeevwni kqmikitqeh
  61 iealldkfgg ehnppsiyle ayeeytskld alqqreqqll eslgngtdfs vsssasmdtv
121 tsssssslsv lpsslsvfqn ptdvarsnpk spqkpivrvf lpnkqrtvvp arcgvtvrds
181 lkkalmmrgl ipeccavyri qdgekkpigw dtdiswltge elhvevlenv pltthnfvrk
241 tfftlafcdf crklifqgfr cqtcgykfhq rcstevplmc vnydqldllf vskffehhpi
301 pqeeaslaet altsgsspsa pasdsigpqi ltspspsksi pipqpfrpad edhrnqfgqr
361 drsssapnvh intiepvnid dlirdqgfrg dggsttglsa tppaslpgsl tnvkalqksp
421 gpqrerksss ssedrnrmkt lgrrdssddw eipdgqitvg qrigsgsfgt vykgkwhgdv
481 avkmlnvtap tpqqlqafkn evgvlrktrh vnillfmgys tkpqlaivtq wcegsslyhh
541 lhiietkfem iklidiarqt aqgmdylhak siihrdlksn niflhedltv kigdfglatv
601 ksrwsgshqf eqlsgsilwm apevirmqdk npysfqsdvy afgivlyelm tgqlpysnin
661 nrdqiifmvg rgylspdlsk vrsncpkamk rlmaeclkkk rderplfpqi lasiellars
721 lpkihrsase pslnragfqt edfslyacas pktpiqaggy gafpvh
```

The present disclosure also provides, in part, a kit for detecting the presence of a BRAF mutation in a biological sample, including a specific binding agent that selectively binds to a BRAF mutation, and instructions for carrying out the method as described herein.

As used herein the term “sample” refers to anything which may contain an analyte for which an analyte assay is desired. In many cases, the analyte is a nucleic acid molecule, such as a DNA or cDNA molecule encoding all or part of BRAF. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “subject in need thereof” is a subject having a cell proliferative disorder, or a subject having an increased risk of developing a cell proliferative disorder relative to the population at large. In some cases, a subject in need thereof has cancer. More preferably, a subject in need thereof has hairy cell leukemia or shows symptoms of suffering from hairy cell leukemia, or is alternatively suspected of having hairy cell leukemia. A “subject” includes a mammal. The mammal can be e.g., any mammal, e.g., a human, primate, bird, mouse, rat, fowl, dog, cat, cow, horse, goat, camel, sheep or a pig. In many cases, the mammal is a human being.

As used herein, a “normal cell” is a cell that cannot be classified as part of a “cell proliferative disorder”. A normal cell lacks unregulated or abnormal growth, or both, that can lead to the development of an unwanted condition or disease. In some cases, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right or abnormal in the body. But signs are defined as things that can be seen or detected by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin, some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the disclosure.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

Other features and advantages of the present disclosure are apparent from the different examples. The provided examples illustrate different components and methodology useful in practicing the present disclosure. The examples do not limit the disclosure.

### EXAMPLES

### Example 1

### Materials and Methods

The following methods described herein were utilized in the examples that follow.

### Tumor Samples

113 HCL patients were studied, including 94 pre-therapy and 19 post-therapy, all with detectable disease. Eleven HCL patients, post-therapy and in complete flowcytometric remission (≦0.1% leukemic cells), 111 patients with other B-cell neoplasms (60 SMZL; 15 SLLU, including 11 HCL-v; 31 chronic lymphocytic leukemias-CLL; 5 unclassifiable CD5-negative mature B-cell neoplasms) and 9 healthy blood donors were also investigated.

Samples from 23 HCL patients and 38 non-HCL patients were previously reported (Tiacci E, et al. N Engl J Med. 2011; 364:2305-2315). Diagnosis of HCL and non-HCL tumors conformed to the WHO-2008 classification (Foucar K., et al. Hairy cell leukaemia. In: Swerdlow S, et al., eds. WHO Classification of Tumours of Haematopoietic and Lymphoid Tissues (4th edition). Lyon: International Agency for Research on Cancer (IARC), Lyon, France; 2008: 188-190; Phis M, et al. Splenic B-cell lymphoma/leukaemia, unclassificable. In: Swerdlow S, et al., eds. WHO classification of tumours of haematopoietic and lymphoid tissues (4th edition). Lyon: International Agency for Research on Cancer (IARC); 2008: 191-193). Patients gave verbal or written consent for the analysis of their sample material.

HCL samples were mostly represented (n=93) by peripheral blood (or bone marrow aspirate diluted by peripheral blood). They were taken before treatment in 74 cases (among which 42/70 with available informations had <30% leukemic cells), after treatment at complete flow-cytometric remission in 11 cases and after treatment with up to 13% residual leukemic cells detected at flow cytometry in 8 cases (of which 5 had only 0.2%-2% leukemic cells). The remaining HCL samples were frozen bone marrow biopsies in 14 cases (all with ≧30% leukemic cells; 12 pre-treatment and 2 post-treatment) and leukemic cells MACS-purified from peripheral blood (≧90%) in 17 cases (8 pre-treatment, 9 post-treatment). The non-HCL tumor samples were represented by peripheral blood or bone marrow aspirate in 90 cases (range of leukemic cells: 2%-97%; 67/80 samples with available informations had ≧30% leukemic cells) and fresh or frozen splenectomy specimens in 21 cases (all SMZL, with ≧30% tumor cells in all 15 cases with available information). Percentages of leukemic cells are reported as fractions of all nucleated cells present in the analyzed sample.

### DNA Extraction

To extract genomic DNA, the QIAamp DNA blood Midi kit (Qiagen) was used for whole-blood samples (from patients) and buffy coats (from healthy donors), and the Puregene core kit A (Qiagen) for MACS-purified leukemic cells and frozen bone marrow sections. Extracted genomic DNA was eluted or resuspended in nuclease-free water.

### Qualitative Allele Specific PCR Amplification (AS-PCR)

To detect the T→A transversion in BRAF exon-15 leading to the V600E replacement, two AS-PCR reactions were developed, each sharing the same reverse primer (5′-GTAACTCAGCAGCATCTCAGGG-3′) (SEQ ID NO:1) and differing for the allele-specific forward primer. The latter was designed to have its 3′-terminus complementary to either the mutated (A) or the wild-type (T) base as follows: 5′-AGGTGATTTTGGTCTAGCTACAGA-3′ (SEQ ID NO: 2, mutated base in bold), 5′-GGTGATTTTGGTCTAGCTACAGA-3′ (SEQ ID NO: 3, mutated base in bold), 5′-AGGTGATTTTGGTCTAGCTACCGA-3′(SEQ ID NO: 4, mutated base in bold), 5′-GGTGATTTTGGTCTAGCTACCGA-3′(SEQ ID NO: 5, mutated base in bold), 5′-AGGTGATTTTGGTCTAGCTACAGT-3′ (SEQ ID NO: 6, wild-type base in bold) and 5′-GGTGATTTTGGTCTAGCTACAGT-3′ (SEQ ID NO: 7, wild-type base in bold). When a negative result is obtained with the mutant-AS-PCR in a given clinical sample, before concluding for the absence of the mutation it is mandatory to perform the wild-type AS-PCR in the same sample as a positive control for the amplifiability of the wild-type BRAF alleles (deriving from the normal cells always contaminating clinical samples, as well as from unmutated or heterozygously mutated tumor cells).

One hundred nanograms of recently extracted genomic DNA (eluted or resuspended in nuclease-free water and quantified through the Quant-iT BR Assay in a Qubit fluorometer—Invitrogen) were amplified in each reaction using 0.5 U of Platinum Taq DNA polymerase (Invitrogen), 1× buffer, 1 mM MgCl₂, 200 nM dNTPs, 100 nM forward (SEQ ID NOs 2 or 3 or 4 or 5 or 6 or 7) and 100 nM reverse primer (SEQ ID NO 1) (from fresh aliquots), in a final reaction volume of 25 μl. Cycling conditions (after the initial denaturation step at 94° C. for 2 minutes) were: 94° C. for 30 minutes, 59° C. for 30 minutes, 72° C. for 20 minutes for 40 cycles, followed by a final elongation at 72° C. for 5 minutes.

Primers were purchased from MWG-Eurofins and PCRs were performed for all samples on a Veriti 96-well Thermal Cycler (Applied Biosystem), starting the cycling program and the lid heating at the same time (as a worse performance of the assay was noted when the lid was pre-heated). For a large subset of samples, PCRs were repeated with the same results in another cycler (Eppendorf Master Cycler), using the same PCR conditions and instrument settings as in the Veriti 96-well Thermal Cycler.

Ten microliters of each PCR product were subjected to electrophoresis through a 2%-agarose gel stained with GelRed (Biotium), a more sensitive dye than ethidium bromide. Images were acquired using a Kodak Gel Logic 100 Imaging System instrument equipped with a UV transilluminator (Carestream Health Inc.).

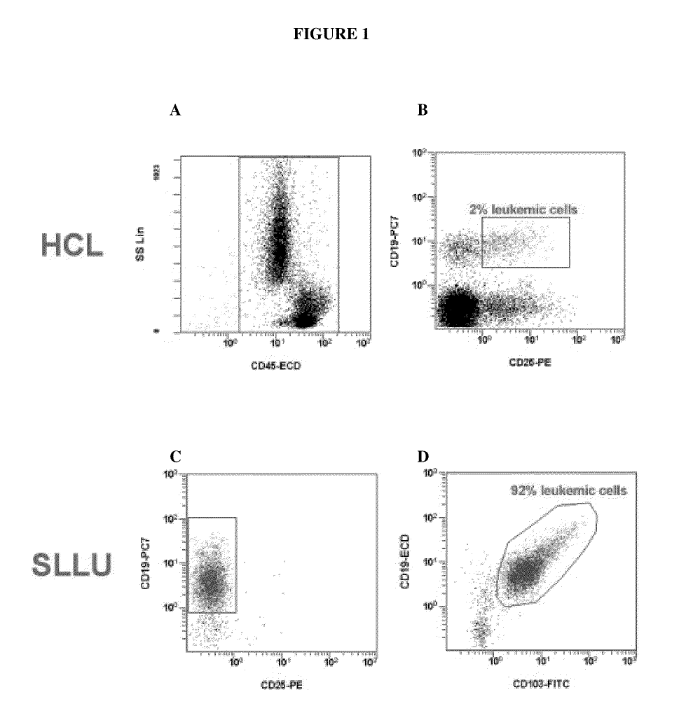

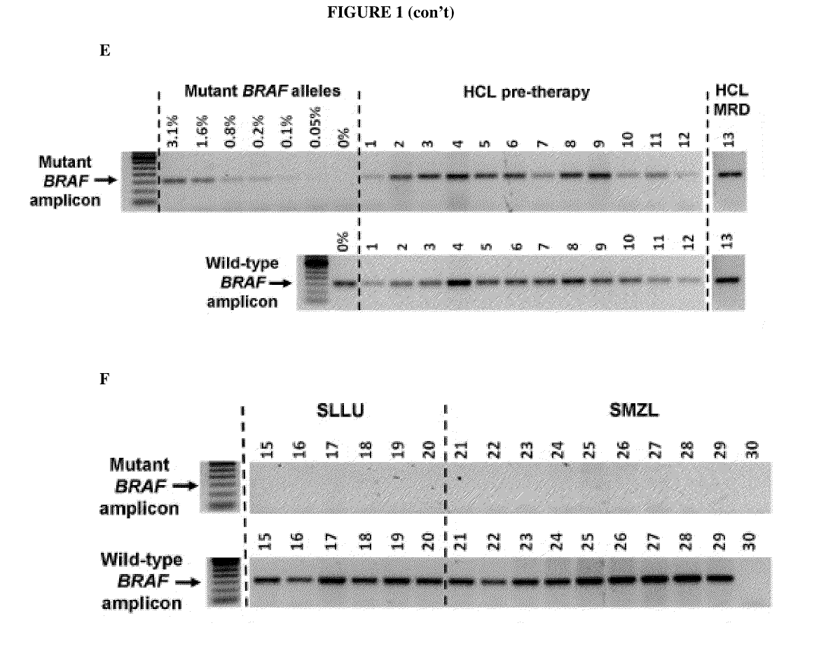

The analytical sensitivity of the AS-PCR assay was estimated to be ≧0.1% mutant BRAF-V600E alleles (FIG. 1) by subjecting to mutant-AS-PCR serial dilutions (100 ng each) of genomic DNA from the homozygously BRAF mutated thyroid carcinoma cell line 8505C (DMSZ—German Collection of Microorganisms and Cell Cultures) mixed with an equal quantity of genomic DNA from a BRAF wild-type sample (buffy coat from a healthy donor), and setting the undiluted cell samples as having 100% BRAF-V600E mutant alleles and 100% BRAF wild-type alleles respectively. Genomic DNA quantification of the samples used for serial dilutions was performed in quintuplicate with the highly precise and DNA-specific Quant-iT BR Assay in a Qubit fluorometer (Invitrogen). It was confirmed the same analytical sensitivity (≧0.1% mutant alleles) when replacing the genomic DNA of 8505C cells with that of MACS-purified (>99%) primary leukemic cells from a HCL patient previously known to harbor a homozygous/hemizygous BRAF-V600E mutation (Tiacci E, et al. N Engl J Med 2011; 364:2305-2315). Serial genomic DNA dilutions such as those described above are to be included in the mutant-AS-PCR along with the clinical samples to positively control the efficient and sensitive amplification of the BRAF-V600E mutant alleles.

### Example 2

### The use of BRAF V600E for the Diagnosis of HCL

The analytical sensitivity of the AS-PCR was first assessed in serial dilutions of DNA from a BRAF-V600E homozygous sample with DNA from a BRAF wild-type sample, and established the lower detection limit to be 0.1% of mutant alleles (FIG. 1), corresponding to 0.2% of diploid tumor cells harboring a clonal heterozygous BRAF-V600E mutation.

Samples from 113 HCL patients (94 pre-treatment; 19 post-treatment with residual or relapsing disease) were then analyzed. All 113 samples tested positive (100% diagnostic sensitivity) (Table 1 and FIG. 1), including 23 samples previously known to harbor BRAFV600E by Sanger sequencing (Tiacci E, et al. N Engl J Med. 2011; 364:2305-2315). Notably, among the newly reported 90 HCL cases, 20 (15 pre-treatment, 5 post-treatment) had only 0.2%-5% leukemic cells and 9 were analyzed at different time-points after the onset of the disease (range 1-26 years).

These findings demonstrate the excellent analytical and diagnostic sensitivity of the test. They also confirm and extend a previous report that BRAF-V600E occurs and persists over the disease course in virtually all HCL cases (Tiacci E, et al. N Engl J Med. 2011; 364:2305-2315), further supporting the view that BRAF-V600E represents the key pathogenetic event in HCL and therefore a new therapeutic target. Indeed, persistence of BRAF-V600E at partial remission or relapse following conventional therapy establishes the rationale for using active-BRAF inhibitors (Flaherty K T, et al. N Engl J Med. 2010; 363:809-819) in this setting. The test may also serve as a new tool (in addition to immunohistochemistry, flow cytometry and immunoglobulin gene rearrangement analysis (Noel P. Leuk Lymphoma. 2011; 52 Suppl 2:62-64; Tallman M S. Leuk Lymphoma. 2011; 52 Suppl 2:65-68)) to assess minimal residual disease (MRD) following therapy, although the clinical relevance of MRD in HCL remains unclear (Tallman M S. Leuk Lymphoma. 2011; 52 Suppl 2:65-68).

The diagnostic specificity of this test was evaluated by analyzing blood samples from 9 healthy donors and 11 HCL patients in complete flow-cytometric remission (≦0.1% leukemic cells) post-therapy, and all tested negative (Table 1 and FIG. 1). Specificity was also assessed in 111 patients with non-HCL chronic B-cell neoplasms. Because the absence or very rare occurrence of BRAF-V600E has been already reported (Tiacci E, et al. N Engl J Med. 2011; 364:2305-2315; Case M, et al. Cancer Res. 2008; 68:6803-6809; Gustafsson B, et al. Leukemia. 2005; 19:310-312; Davidsson J, et al. Leukemia. 2008; 22:1619-1621; Lee J W, et al. Br J Cancer. 2003; 89:1958-1960; Chapman M A, et al. Nature. 2011; 471:467-472) in several B-cell tumors, this study focused on HCL-like disorders that, being rare, have been so far poorly investigated. Therefore, among the 111 cases, 75 patients with splenic marginal zone lymphoma (SMZL) and splenic B-cell lymphoma/leukaemia unclassificable (SLLU) were included, of which 61 previously unreported (Tiacci E, et al. N Engl J Med. 2011; 364:2305-2315). Notably, all 111 cases tested negative (Table 1 and FIG. 1), showing a 100% diagnostic specificity of the assay and further confirming in a larger patient series the absence of BRAF-V600E in HCL-like disorders. Considering that 33 HCL-like cases had ≧40% neoplastic cells and that this test can detect 0.1% mutant alleles, these data also argue against the presence of small BRAF-V600E-mutated subclones (down to 0.5% of a whole leukemic population representing ≧40% of the sample) in HCL-like disorders. This further supports the concept that, among B-cell lymphomas and leukemias, BRAF-V600E is the genetic lesion defining HCL (Tiacci E, et al. N Engl J Med. 2011; 364:2305-2315). Although, collectively, 95 HCL-like disorders (SMZL and SLLU, the latter including 19 HCL-variant) have been analyzed without finding BRAF-V600E in any of them (Tiacci E, et al. N Engl J Med. 2011; 364:2305-2315), the possibility cannot be excluded that this mutation may be rarely found in these and other B-cell neoplasms if a larger number of cases is investigated.

This diagnostic test is especially useful for patients with a low tumor burden in the blood (as typically occurs in HCL) or bone marrow. In this setting, it appears superior to Annexin-A1 immunostaining, that may be difficult to interpret (due to Annexin-A1 expression by myeloid and T cells) unless a technically demanding double staining with a B-cell marker (e.g., PAX5) is performed. The gel-based AS-PCR is considerably more sensitive than a recently described HRMA (High-Resolution-Melting Analysis)-based PCR, which was applied to fewer HCL samples (n=48) containing more (10%) leukemic cells (Boyd E M, et al. Br J Haematol. 2011: Sep. 13, [Epub ahead of print]). Notably, this test detected BRAF-V600E in all blood samples having <10% HCL cells (31/78 samples; 23 pre-therapy; 8 post-therapy) and does not require the expensive instrumentation needed for high-resolution melting analysis (HRMA).

The sensitive, simple and reliable method of the disclosure confirms the constant presence of BRAF-V600E in HCL (Boyd E M, et al. Br J Haematol. 2011: Sep. 13, [Epub ahead of print]) and its absence in HCL-like disorders, adding to the already available diagnostic armamentarium for improving the diagnostic accuracy in HCL and HCL-like disorders.

TABLE 1
Results of AS-PCR in HCL cases and other B-cell neoplasms

| | Leukemic | Number | Mutated | |
|---|---|---|---|---|
| Sample type | cells | of cases | cases | |
| Hairy cell leukemia | | | | |
| blood, pre-treatment | 1%-90% | 74 | 74 | (100%) |
| blood, MRD | 0.2%-13% | 8 | 8 | (100%) |
| blood, complete remission | not detectable | 11 | 0 | (0%) |
| bone marrow biopsies | 30%-80% | 14 | 14 | (100%) |
| purified leukemic cells | ≧90% | 17 | 17 | (100%) |
| Splenic marginal zone | 2%-97% | 60 | 0 | (0%) |
| lymphoma | | | | |
| Splenic lympoma/leukemia | 15%-97% | 15* | 0 | (0%) |
| unclassifiable | | | | |
| Chronic lymphocytic leukemia | 18%-98% | 31 | 0 | (0%) |
| CD5-negative mature B-cell | 15%-97% | 5 | 0 | (0%) |
| neoplasm unclassifiable | | | | |
| Healthy blood donors | not detectable | 9 | 0 | (0%) |

MRD, minimal residual disease.
*Including 11 HCL-variant.

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

We claim:

1. A kit for detecting the presence of a BRAF mutation in a biological sample, comprising,
(a) a forward primer comprising one of the following sequences:
(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,
(SEQ ID NO: 5)
5′-GGTGATTTTGGTCTAGCTACCGA-3′;
(b) a reverse primer comprising the sequence of 5′-GTAACTCAGCAGCATCTCAGGG-3′ (SEQ ID NO: 1) and
(c) instructions for detecting the presence of a BRAF mutation in the biological sample.

2. The kit of claim 1, further comprising a forward primer comprising either the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7).

3. A method, comprising
(a) extracting at least one DNA molecule from a biological sample from a subject (b);
(b) amplifying said DNA molecule by polymerase chain reaction (PCR), using a combination of a forward primer comprising one of the following sequences:
(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,
5′-GGTGATTTTGGTCTAGCTACCGA-3′ (SEQ ID NO: 5), and the reverse primer comprising the sequence of 5′-GTAACTCAGCAGCATCTCAGGG-3′ (SEQ ID NO: 1), wherein the amplification product comprises a mutation in BRAF;
(c) comparing the amplified product of step (b) with a control or wild type amplification product of BRAF;
(d) determining the presence or absence of the mutation in BRAF in the subject; and
(e) diagnosing the subject as having cancer if the mutation is present.

4. The method of claim 3, wherein the control or wild type amplification product of BRAF is amplified using a combination of a forward primer comprising either the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7), and the reverse primer comprising the sequence of 5′-GTAACTCAGCAGCATCTCAGGG-3′ (SEQ ID NO: 1).

5. The method of claim 3, wherein the control or wild type amplification product of BRAF comprises a thymine (T) nucleotide at position 1860 of SEQ ID NO: 8; or
wherein the control or wild type amplification product of BRAF codes for a Valine (Val or V) residue at amino acid residue 600 of SEQ ID NO: 9.

6. The method of claim 3, wherein the DNA molecule is genomic DNA or cDNA.

7. The method of claim 3, wherein mutation in the human BRAF gene is a substitution of an adenine (A) for a thymine (T) nucleotide at position 1860 of SEQ ID NO: 8.

8. The method of claim 3, wherein the mutation in the human BRAF gene encodes for a mutation in the resultant amino acid sequence, wherein a glutamic acid (Glu or E) is substituted for a Valine (Val or V) residue at amino acid residue 600 of SEQ ID NO: 9.

9. The method of claim 1, wherein the biological sample is a tissue sample or a bodily fluid.

10. The method of claim 9, wherein the tissue sample is bone marrow or spleen.

11. The method of claim 10, wherein the bone marrow is bone marrow aspirate diluted by peripheral blood.

12. The method of claim 9, wherein the bodily fluid is whole blood or peripheral blood.

13. The method of claim 1, wherein the cancer is primary or metastatic cancer.

14. The method of claim 1, wherein the cancer is a solid or liquid cancer.

15. The method of claim 1, wherein the cancer is selected from the group consisting of adrenal cortical cancer, anal cancer, bile duct cancer, bladder cancer, bone cancer, brain or a nervous system cancer, breast cancer, cervical cancer, colon cancer, rectral cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), hairy cell leukemia (HCL), non-HCL lymphoid malignancy (hairy cell variant, splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, or splenic lymphoma/leukemia unclassifiable (SLLU)), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, and Wilms Tumor.

16. The method of claim 1, wherein the cancer is hairy cell leukemia (HCL).

17. The method of claim 1, wherein the cancer is a non-HCL lymphoid malignancy.

18. The method of claim 17, wherein the non-HCL lymphoid malignancy is hairy cell variant (HCL-v), splenic marginal zone lymphoma (SMZL), chronic lymphocytic leukemia (CLL), splenic diffuse red pulp small B-cell lymphoma (SDRPSBCL), prolymphocytic leukemia, low grade lymphoma, systemic mastocytosis, or splenic lymphoma/leukemia unclassifiable (SLLU).

19. A kit for detecting the presence of a BRAF mutation in a biological sample, comprising,
(a) a forward primer comprising one of the following sequences:
(SEQ ID NO: 2)
5′-AGGTGATTTTGGTCTAGCTACAGA-3′,
(SEQ ID NO: 3)
5′-GGTGATTTTGGTCTAGCTACAGA-3′,
(SEQ ID NO: 4)
5′-AGGTGATTTTGGTCTAGCTACCGA-3′,
(SEQ ID NO: 5)
5′-GGTGATTTTGGTCTAGCTACCGA-3′;
(b) a reverse primer comprising the sequence of 5′-GTAACTCAGCAGCATCTCAGGG-3′ (SEQ ID NO: 1) and
(c) instructions for carrying out the method of claim 3.

20. The kit of claim 19, further comprising a forward primer comprising either the sequence 5′-AGGTGATTTTGGTCTAGCTACAGT-3′ (SEQ ID NO: 6) or the sequence 5′-GGTGATTTTGGTCTAGCTACAGT-3′ (SEQ ID NO: 7).
